(19) United States
(12) Patent Application Publication
(10) Pub. No.: US 2014/0193346 A1
(43) Pub. Date: Jul. 10, 2014

(54) HEALTH SUPPLEMENT

(71) Applicant: DRT LABORATORIES LLC, Airmont, NY (US)

(72) Inventor: Ronit Arginteanu, Cresskill, NJ (US)

(73) Assignee: DRT LABORATORIES LLC, Airmont, NY (US)

(21) Appl. No.: 13/735,298

(22) Filed: Jan. 7, 2013

(51) Int. Cl. A61K 31/365; A61K 31/353; A61K 31/09; A61K 31/045; A61K 33/42; A61K 31/7008; A61K 36/30; A61K 31/165; A61K 31/201; A61K 31/341

(52) U.S. Cl. CPC A61K 31/365; A61K 31/201; A61K 31/353; A61K 31/09; A61K 31/341; A61K 33/42; A61K 31/7008; A61K 36/30; A61K 31/165; A61K 31/045
USPC 424/49; 514/560; 514/456; 514/469; 514/721; 514/461; 514/473; 424/605; 514/161; 514/62; 424/725; 514/627

(57) ABSTRACT

Herbal composition and method for the treatment of inflammatory pain associated with tendonitis, bursitis, arthritis, carpal tunnel syndrome, tennis elbow, plantar fasciitis, sports injuries, lower back pain and overexertion, gingivitis and muscle aches, that is compliant with HPUS monographs and/or over-the-counter FDA topical analgesic monograph requirements.

### FIELD OF THE INVENTION

This invention is a novel herbal composition comprising extracts from a selection of plants that have anti-inflammatory properties, and the method of application. The composition, when applied to the site of inflammation, reduces inflammatory pain.

### BACKGROUND OF THE INVENTION

Phytotherapy is a thriving medical methodology that uses plants to treat people, facilitating the healing process within the framework of holistic medicine. With its roots in the past, it remains relevant and meaningful in the present. The ecological integration of plant activity with human physiology offers the potential for facilitating the healing process.

Inflammation is a complex immune response of living tissue to injury. An inflammatory response is essential for life and preservation of function, but can also be the result of significant tissue damage and loss of function. The inflammatory process is triggered whenever tissues are injured, and injury may include a physical injury, infection, allergies, etc. The typical signs or symptoms of acute inflammation are redness (erythema), swelling (edema), heat and pain. Inflammation occurs in response to many different causes, including an injury and is initiated by the release of chemical mediators into the extracellular fluid. Sources of these (chemical mediators?) include injured tissue cells, phagocytes, lymphocytes, mast cells and blood proteins such as histamine, kinins, prostaglandins and lymphokines. They are all vasodilators; as blood flow increases to the area, blood congestion ensues, resulting in redness and heat in the area.

Just as inflammation covers a complex series of reparative and protective responses to tissue injury (regardless of whether caused by infection, autoimmune stimuli or mechanical injury), anti-inflammatory substances used to treat inflammation address similar mechanisms, albeit by different mediators. Currently used anti-inflammatory agents such as Nonsteroidal anti-inflammatory drugs “NSAIDs” operate by inhibiting cyclooxygenase and therefore inhibit prostaglandin synthesis (the manufacture of lipid compounds that mediate biological processes such as inflammation). Free radical scavenging agents also play a role in inflammation because liberation of such radicals damages tissue during the inflammatory process. Flavonoids and other phenolics are believed to act by preventing either the generation or the action of free radicals. Studies have shown that quercetin, a plant-derived flavonoid readily found in nature, inhibits the action of liposygenase on arachidonic acid, the release of histamine from mast cells and basophils, and the generation of superoxide anion.

Part of the polyphenol family, flavonoids are valued pharmacologically for their anti-inflammatory activities (such as quercetin). About 8,000 naturally occurring plant phenolics have been identified, half of which are flavonoids, contributing significantly to the color, taste and flavor of many herbs, foods and drinks. Many phenolic molecules are effective antioxidants and free radical scavengers, in addition to being the basis for most flower colors.

Flavonoids have been designated as nature's biological response modifiers because they can change or alter the body's reaction to allergens and viruses. Flavonoids are often found in the cell sap, flowers and fruits of plants. Also synthesized in the body from the amino acid phenylalanine, flavonoids can be classified into three chemical categories: flavones (derived from 2-phenylchromen-4-one (2-phenyl-1,4-benzopyrone) structures), isoflavonoids (derived from 3-phenylchromen-4-one [3-phenyl-1,4-benzopyrone] structures), and neoflavonoids (derived from 4-phenylcoumarine [4-phenyl-1,2-benzopyrone] structures). Both in vitro and in vivo studies attest to flavonoid's potential use in modern medicine.¹ A comprehensive review by Middleton and Kandaswami of the impact of flavonoids on immune system function and the inflammatory response illuminated a wide range of effects.² ¹Middleton E. The flavonoids as potential therapeutic agents. In: Kimball SE, ed. Immunopharmaceuticals. Boca Taton, Fla.: CRC Press, 1995. ²Middleton E. Kandaswami C. Effects of flavonoids on immune and inflammatory cell functions. Biochemical Pharmacology 1992 Mar. 17; 43(6): 1167-79.

Flavonoids may help provide protection against certain diseases by contributing, along with antioxidant vitamins and enzymes, to the total antioxidant defense system of the human body. Some studies have shown that flavonoid intake is inversely related to mortality from coronary heart disease and to the incidence of heart attacks as well as anti-inflammatory activity against arthritis. Several in vitro studies and a few in vivo animal studies have shown that various flavonoids, such as quercetin, possess anti-inflammatory properties.³ Recent press indicates flavonoids are helpful as antioxidants, potentially slowing down the aging process; however, more epidemiological studies remain necessary on this topic. ³Calder P C, Albers R, Antoine J M, et al.: Inflammatory disease processes and interactions with nutrition. British Journal of Nutrition 2009; 101 Suppl 1: S1-45; Garcia-Lafuente A, Guillamon E, Villares A, Rostagno M A, Martinez J A: Flavonoids as anti-inflammatory agents: implications in cancer and cardiovascular disease. Inflammatory Response 2009; 58(9): 537-52.

The anti-inflammatory properties of flavonoids, may affect prostaglandin synthesis and arachidonic acid metabolism, enzyme inhibition and antioxidant activities. Some flavonoids inhibit prostaglandin synthesis, in contrast to others which actually stimulate prostaglandin production. Those flavonoids that inhibit prostaglandin synthetase (PGS) suppress inflammation. Furthermore, leukotrines are products of arachidonic acid that are produced by the 5-lipoxygenase pathway, and are involved in the inflammatory process and can be inhibited by flavonoids. Quercetin has been shown to actually inhibit lipoxygenase. Histamine is an organic nitrogen compound in the human body that is involved in local immune responses and can also trigger an inflammatory response. Histamine initiates the swelling that is a product of a sudden reaction. In human cell culture studies, Quercetin, Apigenin and other flavonoids have been shown to inhibit histamine release, thereby inhibiting the inflammatory response. Quercetin's selective inhibition of pro-inflammatory arachidonic acid metabolites may assist in explaining its anti-inflammatory and antiallergic actions.⁴ ⁴ Hertog M G, Feskens E J, Hollmn P C, et al. Dietary antioxidant flavonoids and risk of coronary heart disease: the Zutphen Elderly Study. Lancet 1993 Oct. 23; 342 (8878): 1007-11.

Additionally, free radicals, or active oxygen species, can cause serious damage in inflammation. These species emanate from superoxide anions liberated from stimulated phagocytic cells. Most flavonoids are antioxidants and efficient scavengers of free radicals. They remove these free radicals, and even suppress their formation by binding heavy metal ions that are believed to catalyze the production of free radicals.

Moreover, flavonoids inhibit an enzyme called phosphodiesterase, which catalyzes the breakdown of cyclic adenosine monophosphate (cAMP). cAMP is a vital regulatory molecule in cellular activity, triggering a range of intracellular effects by activating different enzymes in the human body. High cAMP levels are anti-inflammatory. In the presence of inflammation, drugs that elevate intracellular cAMP levels have some anti-inflammatory action. Flavonoids can inhibit the breakdown of cAMP, which may be another reason for their anti-inflammatory effects.

Lastly, flavonoids appear to have an important advantage over NSAIDs in that they are largely free of adverse effects. NSAIDs work in part by inhibiting the synthesis of prostaglandins, not only those that cause inflammation, but also the cytoprotective prostaglandins that help prevent ulceration of the gastric mucosa. Flavonoids do not cause gastric ulceration and, as a class, are remarkably free of side effects.

The molecular structure of the flavone backbone (2-phenyl-1,4-benzopyrone) such as Apigenin and Luteolin:

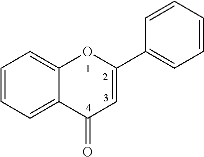 [SMILES: O=c1cc(-c2ccccc2)oc2ccccc12]

The molecular structure of Quercetin (3-hydroxy-2-phenyl-1,4-benzopyrone)

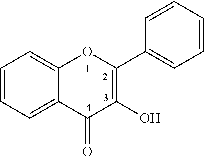 [SMILES: O=c1c(O)c(-c2ccccc2)oc2ccccc12]

Very little doubt can remain that flavonoids have profound effects on the function of inflammatory cells, as confirmed by a large variety and number of in vitro and in vivo studies. It is highly likely that these phytonutrients exhibit a vital, serious effect on secondary cell systems that comprise the inflammatory response.

Similarly, Lignans are plant hormones known as phytoestrogens that are believed to have an anti-inflammatory effect on the human body. A phytoestrogen is a plant constituent that possesses estrogenic activity. Phytoestrogens are not precursors of human hormones, but rather they interact with human estrogen receptors to elicit various responses. For example, laboratory studies have demonstrated the ability of lignans to bind to the nuclear estrogen receptor, activate a response and promote growth of estrogen dependent cells in culture.⁵ Plant lignans come from sources such as flax seed, whole grain cereals, rye, legumes, seeds and nuts. Several hundred individual lignans have been discovered. Diets high in lignans have demonstrated in animals a direct anti-inflammatory effect due to the known antioxidant properties.⁶ Investigations on the effects of plant lignans reveal that the isolated lignan derivatives contribute strongly to anti-inflammatory activity by intervening against inflammatory markers. Isolated pure lignans inhibit the production of major systemic markers of inflammation, including pro-inflammatory eicosanoids, cytokines and platelet-activating factor. These effects have been demonstrated in animals, healthy volunteers and in patients diagnosed with systemic lupus erythematosus and rheumatoid arthritis. Furthermore, regular lignan intake can help retard the production of atherosclerosis by reducing inflammatory responses. ⁵Miksicek R J. Commonly occurring plant flavonoids have estrogenic activity. Molecular Pharmacology 1993 July; 44(1): 37-43. ⁶Kinniry P, Amrani Y, Vachani A, et al: Dietary Flaxseed Supplementation Ameliorates Inflammation and Oxidative Tissue Damage in Experimental Models of Acute Lung Injury in Mice. American Society for Nutrition 2006; 136: 1545-1551, June 2006.

The following represent the molecular structure of some Lignan precursors:

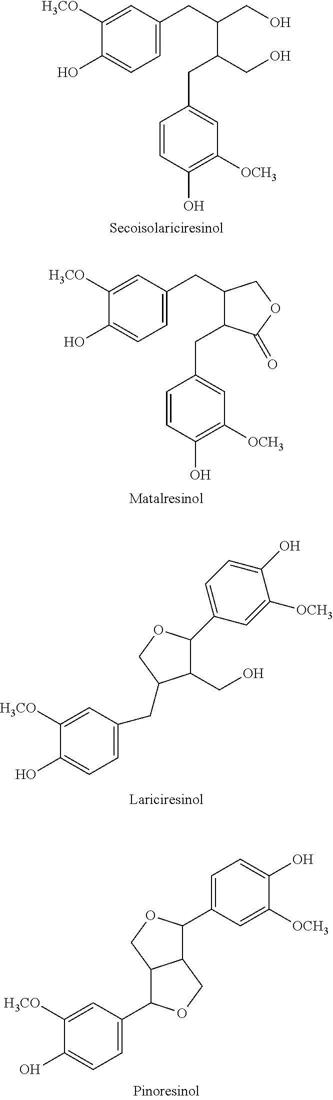 [SMILES: COc1cc(CC(CO)C(CO)Cc2ccc(O)c(OC)c2)ccc1O ; COc1cc(CC2COC(=O)C2Cc2ccc(O)c(OC)c2)ccc1O ; COc1cc(CC2COC(c3ccc(O)c(OC)c3)C2CO)ccc1O ; COc1cc(C2OCC3C(c4ccc(O)c(OC)c4)OCC23)ccc1O]

Plants are also a source of fatty acids, which they occur mostly as fats or lipids. Considerable quantities of lipids occur in the seeds or fruits of many plants, providing a stored form of energy to use during germination. Most fatty acids come from the diet. In particular, two highly unsaturated fatty acids known as linoleic acid and linolenic acid cannot be synthesized from precursors in the body. These are known as the essential fatty acids which must be acquired by humans by consuming plants or eating the meat of animals that have consumed these plant fats. Examples are omega-3 fatty acids (linoleic acid) and omega-6 fatty acid (gamma linolenic acid).

Finally, Linoleic acid is a polyunsaturated fatty acid (C₁₈H₃₂O₂) of the omega-6 series. Linoleic and another fatty acid, gamma-linolenic, or gamolenic (GLA), produce compounds called prostaglandins. Prostaglandins are substances that are found in every cell, are needed for the body's overall health maintenance, and must be replenished constantly. Some of the plants with seeds that contain GLA have been used as folk remedies for hundreds, if not thousands, of years. However, the discovery of GLA and of these seed oils as a source of GLA is much more recent.

Research in the 1980s found that prostaglandins play an active role in making smooth muscles contract, controlling inflammation and body temperature, and in other body functions. Since GLA was known to be a building block for some prostaglandins, it was reasoned that GLA might be help treat human disease. While GLA is widely touted for its health benefits, research on its effectiveness in human diseases is still at an early stage. Data from a number of studies and trials have demonstrated the potential beneficial effects of different linoleic acids on cancer, atherosclerosis, inflammation and changes in body composition. Despite the increasing knowledge about linoleic acids' implications on health, the mechanism of action of these fatty acids is not completely understood. Suggestions made recently that the anti-carcinogenic and anti-atherosclerosis effects of linoleic acids stem from their anti-inflammatory properties is of vital importance. Because the physiology of many diseases involve an inflammatory response, further investigation in this area is of growing interest.

The following represent the molecular structure of a fatty acid:

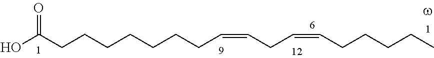 [SMILES: CCCCC/C=C\C/C=C\CCCCCCCC(=O)O]

The herbal composition of the present invention contains phytonutrients such as flavonoids, lignans and fatty acids that collectively form an effective method for treatment of inflammatory diseases.

### DETAILED DESCRIPTION OF INVENTION

The detailed description provides examples and embodiments of the invention, but they are provided for illustration only and are not to limit the disclosure in any way. The scope of the invention contains additional examples and embodiments which will be apparent to one with skill in the art.

It must be noted that, as used in the subject specification, the singular forms “a,” “an” and “the” include plural aspects unless the context clearly dictates otherwise. Thus, for example, reference to “an extract” includes a single extract, as well as two or more extracts; and so forth.

Unless otherwise defined, all technical and scientific terms used herein have the same meaning as commonly understood by one of ordinary skilled in the art to which the invention belongs.

The term “extract” as used herein should be taken in the broadest possible sense. “Extracts” may include tinctures, triturations, fluid extracts or solid extracts, tar and infused oils, for example.

The term “inflammatory disorder or disease or condition” as used herein refers to a disease or condition characterized by chronic inflammation including but not limited to rheumatoid arthritis, osteoarthritis, psoriatic arthritis, tendonitis, bursitis, carpal tunnel syndrome, plantar fasciitis, psoriasis, gingivitis, sports injuries, back pain, overexertion and muscle pain.

The term, “therapeutically effective amount” as used herein means an amount of compound or composition sufficient to significantly induce a positive modification in the condition to be regulated or treated, but low enough to avoid side effects, if any within the scope of sound medical judgment. The therapeutically effective amount of the compound or composition will vary with the particular condition being treated, the age and physical condition of the end user, the severity of the condition being treated/prevented, the duration of the treatment, the nature of concurrent therapy, the specific compound or composition employed, the particular pharmaceutically acceptable carrier utilized and like factors.

The term “subject” as used herein refers to any animal having symptoms associated with or caused by viral infections, which requires treatment with the herbal composition. the subject may be an animal, such as a mammal, preferably a human, or may be a non-human primate or non-primates such as used in animal model testing. While it is particularly contemplated that the herbal composition is suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids and ungulates.

Generally, the terms “treating,” “treatment” and the like are used herein to mean affecting a subject, tissue or cell to obtain a desired pharmacological and/or physiological effect. The effect may be prophylactic in terms of completely or partially preventing evidence of an inflammatory condition, and/or may be therapeutic in terms of a partial or complete cure of an inflammatory condition or disease. “Treating” as used herein covers any prevention or treatment of an inflammatory condition or disease in an animal such as a mammal, more particularly a human, and includes:
a. preventing the inflammatory condition or disease referred to above from occurring in a subject that may be predisposed to such symptoms, but has not yet developed the condition;
b. inhibiting the development of the inflammatory condition or disease referred to above; or
c. alleviating, relieving or ameliorating the effects, i.e. the cause or regression of the inflammatory condition or disease.

The herbal composition may include one or more different kinds of extracts. For example, extracts in the form of a tincture, a tar and an infused oil. Furthermore, the different herbs may be processed for form extracts by different means. Similarly, the herbal composition may comprise a mixture of two or more types of extracts of a particular herb.

Tinctures may be formed from water, alcohol or ethanol-based infusions and decoctions. Alternative bases for the extract may include acetracts (to form a vinegar extract), glycetracts (to form a glycerine extract), mels (to form a honey extract), oxymels (to form a honey and vinegar extract) or syrups (to form a sugar extract).

Tinctures are prepared by methods well known in the art. Briefly, however, a fresh plant tincture is made by first obtaining an herb dried by air as known in the art. The dried herbs are then further dried in an oven at a temperature of between 60 degrees C. and 70 degrees C. The oven dried herbs are then suspended in a solution in a storage container. In a preferred form, the solution is a mixture of alcohol and water. To make up a 1:3 tincture, 50 grams of oven dried herb is suspended in 150 ml of the solution (weight of herb×3 to get a 1:3 ration) in a storage container. The storage container is allowed to stand for 4 to 6 weeks and is shaken periodically. The mixture is then filtered and the solution withdrawn provides the tincture used. Alternatively, after identification of the plant, it is sorted, chopped, weighed and put to macerated or pounded to a pulp. The pulp is then subjected to pressure, the expressed juice gathered in a clean container. the juice is then mixed with equal parts by weight of diluent (water, alcohol, etc.). The mixture is allowed to stand for eight days or more, after which the liquid is decanted and filtered. The final product is a succus that can be suitably stabilized to prevent chemical degradation and microbial contamination.

Other methods of extracting herbs are well known in the field. For example, cold percolation techniques may be used to prepare tinctures.

The tinctures may also be purchased from herbal suppliers such as MediHerb Party Ltd, 124 McEvoy Street, Warwich, Queensland 4370, Australia or from the American Association of Homeopathic Pharmacists, 4332 S.E. Logus Road, Milwaukie, Oreg. 97222.

An infused oil may be made by infusing or macerating the herb in an oil. Infusing or macerating an herb in an oil solubilises the herb, making the resulting infused oil particularly suitable for topical application. The oil used to prepare the infused oil may be a fixed or vegetable oil. A fixed or vegetable oil. A fixed oil is a non-volatile animal or plant based oil.

Examples of fixed and vegetable oils include almond oil, coconut oil, corn oil, peanut oil, olive oil, sesame oil, soybean oil, castor oil, wheat germ oil, cod liver oil, borage oil, pumpkin oil, safflower oil, and pumpkin oil.

Cold pressed oils are particularly preferred. Methods for preparing cold pressed oils are well known in the field. Generally, the process involves a simple crushing and filtering process.

The process of infusion generally involves the following steps: allow the herb plant material to wilt overnight; place the wilted herb plant material in a container, such as a jar, and cover with the chosen oil; cover jar with a lid and shake gently to remove any air bubbles around the herb plant material; remove the lid from the jar and cover the jar with muslin to allow moisture to escape but to prevent foreign objects such as dust and insects to fall into the oil and herb plant material mixture; leave jar in lukewarm place where light is excluded for 2 to 3 weeks; strain oil and herb plant material mixture through muslin or filter paper into a second jar and leave to stand for a week to allow any water from the herb plant material to sink to the bottom; decant off the oil and leave it to stand again to make sure no moisture remains below the oil; decant and store infused oil in a cool place in a dark glass vessel.

To make a stronger oil infusion, a fresh lot of herbs is added to the infused oil and the above process is repeated. Dried herbs may be used in the same way. This general process of infusion may be used to infuse a variety of oils. A tar is a very strong decoction, which is further reduced by evaporation.

Triturations are prepared by methods well known in the art. Briefly, however, a solid substance such as magnesium phosphate is prepared by triturating the dry crude substance with an excipient such as lactose. The apparatus required are an unglazed porcelain mortar, pestle, spatula, measuring tile, stop-clock and balance. The process of trituration generally involves the following steps: One part of the drug substance and 9 or 99 parts of sugar milk is taken; the drug is taken in a porcelain mortar that is unglazed by rubbing with wet sand; the sugar of milk is divided into three parts in the ratio of 1:3:5 or 11:33:55, on a measuring tile; the process of trituration takes one hour.
a. The entire process is divided into three stages. Each stage consumes twenty minutes. In each stage, one part of sugar of milk is added.
b. Each stage is divided into two sub-stages that consume ten minutes each. The process carried out in the first ten minutes of each stage is repeated for the next ten minutes.
c. Each sub-stage of ten minutes includes grinding or pulverizing for six minutes; and scraping and mixing for four minutes.

Trituration is done for six minutes with pestle fully pressed down with firm grip and thumb on top. It is firmly moved in anticlockwise direction for a right-handed person, in a circular fashion, going from center to periphery and back from periphery to center without lifting the hand, movement being at wrist joint. The mixture is then thoroughly scraped from the walls of the mortar with the spatula and mixed uniformly together.

The extracts may be prepared from any part of the herb plant such as, for example, foliage, leaves, the root, flowers, bark, stems or rhizome, seeds and fruit. However, particular parts of the herb plant are usually used to prepare the extracts.

A Poke extract is usually made from the root. In a preferred embodiment of the invention, the Poke extract is prepared as a tincture. The herbal composition preferably comprises from about 0.0001% to 10%, more preferably from about 0.01 to 0.0001% of Poke extract.

A Borage extract is usually made from the flower or leaf. In a preferred embodiment of the invention, the Borage extract is prepared as infused oil. The herbal composition preferably comprises from about 1% to 30%, more preferably from 15% to 25%.

A Pumpkin extract is usually made from the foliage or seed. In a preferred embodiment of the invention, the Pumpkin extract is prepared as infused oil. The herbal composition preferably comprises from about 1% to 40%, more preferably from 15% to 25%.

A Sunflower extract is usually made from the foliage or seed. In a preferred embodiment of the invention, the Sunflower extract is prepared as infused oil. The herbal composition preferably comprises from about 10% to 45%, more preferably from 15% to 40%.

A safflower extract is usually made from the flower, leaf or seed. In a preferred embodiment of the invention, the Borage extract is prepared as a tincture. The herbal composition preferably comprises from about 10% to 20%, more preferably from 5% to 20%.

The herbal composition may comprise from about 0.001% to about 10% of each of the aforementioned analgesics.

In addition to the above-mentioned herb extracts, the herbal composition may further comprise one or more additional herb extracts, which may also assist in preventing and/or treating inflammatory activity. Examples include herb extracts of one or more of Grape Seed (Vitis Vinifera, Flos), Fox Grape (Paris Quadifolia), Wild Thyme (Thymus Serpyllum), Clove Tree (Eugenia Caryophyllus), Juniper (Juiperus Sabina Officinalis), Yellow Sweet Clover (Melilotus Officionalis), Chamomile (Chamomilla), Calendula (Calendula Officinalis), Rosemary (Ledum Palustre/Rosmarinus Officinalis), Lavendar (Agnus Castus), Eucalyptus (Eucalyptus Globulus), Daisy (Bellis Perennis), Wofsbane (Aconditum Napellus), St. John's Wort (Hypericum Perforatum), and Black Cohosh (Cimicifuga Racemosa).

The herbal composition may comprise from about 0.001% to about 10% of each of the above-mentioned additional herb extracts.

The herbal composition may conveniently be administered together with one or more carriers. Carriers include substances that are useful in preparing a formulation comprising the herbal composition, may be in co-administration with the herbal composition while allowing the individual components to perform their intended function, and are generally safe, non-toxic and are neither biologically or otherwise undesirable. Moreover, carriers will include those suitable for veterinarian use as well as human use. Examples of carriers include dispersing agents, suspending agents, emulsifying agents, stabilising agents, wetting agents, binding agents, lubricants, disintegrants, solvents, media, delay agents, fillers, aqueous and oily bases, non-aqueous vehicles, i.e. edible oils and the like.

Additionally, the herbal composition may contain preserving agents, sweetening agents, coloring agents, flavoring agents, thickening and/or gelling agents, buffers and the like. Those skilled in the art will be able to identify further additives that may be desirable for cosmetic reasons, palatability or shelf life, for example.

Furthermore, the herbal composition may be converted into customary formulations. Examples of formulations include, but are not limited to, solutions, emulsions, suspensions, powders, granules, natural and synthetic materials impregnated with the individual components of the herbal composition, pills, capsules, tablets, cachets, pastilles, lozenges, bolus, electuary, pastes, gels, ointments, creams, plasters, washes, lotions, transdermal patches, bandages and dressings including hydrosiolloid dressings, enemas, suppositories, pessaries, sprays (atomiser or aerosol), mouthwashes, toothpastes, syrups and/or elixers. In a preferred form of the invention, the herbal composition is converted into a formulation suitable for topical administration including at the site of inflammation.

One embodiment of the invention is compliant with the Homeopathic Pharmacopeia of the United States monograph (HPUS) and another embodiment will include at least one analgesic approved by the Federal Drug Administration for over the counter usage, including menthol, capsaicin, camphor, glucosamine sulphate, comfrey, and/or salicylic acid.

One preferred embodiment of the present invention includes the combination of extracts from poke root, pumpkin, sunflower, borage, safflower, grape seed, chamomile, calendula, eucalyptus, and black cohosh.

The choice of carriers and/or additives may be limited to some extent by the intended dosage form of the herbal composition and the mode of administration of the herbal composition.

Formulations comprising the herbal composition may be produced by a number of techniques standard in the art, for example, by mixing the herb extracts with one or more carriers and/or additives until all components are well blended. The formulations may be stored in suitable containers, receptacles, vessels and the like.

As will be appreciated, the dose administered, the period of administration and the general administration regime may differ between subjects depending on variables such as their tolerance to certain active ingredients, weight, metabolism, the mode of administration chosen, the severity of the symptoms, and the age and/or general health of the subject. In general, when the herbal composition is converted to a formulation suitable for topical administration, the general administration regime for topical treatment is application to the affected areas about 2 to 4 times daily. Topical treatment may also be supplemented with an oral administration.

Treatment is preferably commenced before or at the time the inflammatory activity develops and preferably continues until the inflammatory process discontinues.

The invention claimed is:

1. A herbal composition comprising extracts from Poke Root (Phytolacca Decandra), Pumpkin (Curcurbita Pepo), Sunflower, Borage and Safflower.

2. A herbal composition of claim 1 further comprising an extract of at least one of the following Grape Seed (Vitis Vinifera, Flos), Fox Grape (Paris Quadifolia), Wild Thyme (Thymus Serpyllum), Clove Tree (Eugenia Caryophyllus), Juniper (Juiperus Sabina Officinalis), Yellow Sweet Clover (Melilotus Officionalis), Chamomile (Chamomilla), Calendula (Calendula Officinalis), Rosemary (Ledum Palustre/Rosmarinus Officinalis), Lavender (Agnus Castus), Eucalyptus (Eucalyptus Globulus), Daisy (Bellis Perennis), Wofsbane (Aconditum Napellus), St. John's Wort (Hypericum Perforatum), and Black Cohosh (Cimicifuga Racemosa).

3. A herbal composition according to claim 2, further comprising at least one magnesium phosphate compound from a group complying with an Homeopathic Pharmacopeia of the United States (HPUS) monograph.

4. A herbal composition according to claim 2, further comprising at least one analgesic selected from Food and Drug Administration over-the-counter analgesic monograph including capsaicin, menthol, camphor, glucosamine sulphate, comfrey, and salicylic acid.

5. A herbal composition according to claim 2 wherein the composition is administered with one or more carriers.

6. A herbal composition according to claim 2 wherein the composition is created in a form selected from a group consisting of a gel, cream, lotion, solution, powder, ointment, hydrocolloid dressing, transdermal patch and bandage.

7. A herbal composition according to claim 2 wherein the composition is designed to be administered topically.

8. A herbal composition according to claim 2 wherein the composition is created as a liquid for use as mouthwash.

9. A herbal composition according to claim 1, further comprising at least one magnesium phosphate compound selected from a group complying with an Homeopathic Pharmacopeia of a United States (HPUS) monograph.

10. A herbal composition according to claim 1, further comprising at least one analgesic selected from a Food and Drug Administration over-the-counter analgesic monograph including capsaicin, menthol, camphor, glucosamine sulphate, comfrey, and salicylic acid.

11. A herbal composition according to claim 1, wherein a formulation is manufactured in a medicine form to treat or prevent inflammation.

12. A herbal composition according to claim 1, wherein a formulation is manufactured with an agent.

13. A herbal composition according to claim 1 wherein the composition is created as a toothpaste.

14. A method of treating or preventing inflammation comprising:
selecting a herbal composition comprising extracts from Poke Root (Phytolacca Decandra), Pumpkin (Curcurbita Pepo), Sunflower, Borage and Safflower;
applying the composition by topically administering to a site of inflammation.

15. A method of treating or preventing inflammation according to claim 14 further comprising: including at least one additional extract chosen from of the following: Grape Seed (Vitis Vinifera, Flos), Fox Grape (Paris Quadifolia), Wild Thyme (Thymus Serpyllum), Clove Tree (Eugenia Caryophyllus), Juniper (Juiperus Sabina Officinalis), Yellow Sweet Clover (Melilotus Officionalis), Chamomile (Chamomilla), Calendula (Calendula Officinalis), Rosemary (Ledum Palustre/Rosmarinus Officinalis), Lavendar (Agnus Castus), Eucalyptus (Eucalyptus Globulus), Daisy (Bellis Perennis), Wofsbane (Aconditum Napellus), St. John's Wort (Hypericum Perforatum), and Black Cohosh (Cimicifuga Racemosa).

16. A method of treating or preventing inflammation according to claim 14 wherein the herbal composition further comprises including at least one magnesium phosphate. compound selected from a group complying with an Homeopathic Pharmacopeia of a United States (HPUS) monograph.

17. A method of treating or preventing inflammation according to claim 14 wherein the herbal composition further comprises at least one analgesic selected from a Food and Drug Administration over-the-counter analgesic monograph including capsaicin, menthol, camphor, glucosamine sulphate, comfrey, and salicylic acid.

18. A method of treating or preventing inflammation according to claim 15 further comprises including at least one magnesium phosphate. compound selected from a group complying with an Homeopathic Pharmacopeia of a United States (HPUS) monograph.
